(19) United States
(12) Patent Application Publication
(10) Pub. No.: US 2025/0000539 A1
(43) Pub. Date: Jan. 2, 2025

(54) Minimally Invasive Carpal Tunnel Release Device and Method

(71) Applicant: Guilherme Giusti, Ponte Vedra Beach, FL (US)

(72) Inventor: Guilherme Giusti, Ponte Vedra Beach, FL (US)

(21) Appl. No.: 18/754,460

(22) Filed: Jun. 26, 2024

Related U.S. Application Data
(60) Provisional application No. 63/524,301, filed on Jun. 30, 2023.

(51) Int. Cl. A61B 17/32; A61B 1/317; A61B 17/34

(52) U.S. Cl. CPC A61B 17/320036; A61B 1/317; A61B 17/3421

(57) ABSTRACT

A minimally invasive carpal tunnel release device and method involves an outer cannula and an inner cannula insert which are collectively inserted into a patient's wrist and positioned in the patient's carpal canal. The outer cannula includes a dorsal groove that, once properly positioned and once the inner cannula insert is removed from within the outer cannula, receives and seats the transverse carpal ligament. The inner cannula insert includes a blade projection, either fixed or retractably deployed, which is used to sever the transverse carpal ligament seated within the dorsal groove of the outer cannula when the inner cannula insert is reinserted within the outer cannula. Releasing the transverse carpal ligament in this manner reduces the risk of damaging the median nerve while the transverse carpal ligament is being released. The outer cannula can further include a probe at its distal end for palpating the transverse carpal ligament.

### CROSS-REFERENCE TO RELATED APPLICATION

This application is a non-provisional of, and claims the benefit of, U.S. Provisional Application Ser. No. 63/524,301, filed Jun. 30, 2023, which is incorporated herein by reference in its entirety.

### FIELD OF THE INVENTION

The present invention relates generally to devices for repairing injuries associated with carpal tunnel syndrome, and more particularly relates to a minimally invasive carpal tunnel release device and method.

### BACKGROUND OF THE INVENTION

Carpal tunnel syndrome (CTS) is a common nerve disorder that affects the hands. It occurs when the median nerve, which runs from the forearm to the hand, becomes compressed as it passes through the carpal tunnel in the wrist. The resulting symptoms can include pain, numbness and tingling, and thumb weakness.

Treatment options for CTS include conservative measures such as activity modification, night splinting, and nonsteroidal anti-inflammatory medication (NSAIDs). Sometimes, steroid injections can be used as a therapeutic treatment option or for diagnostic purposes. Eventually, carpal tunnel surgery is indicated for moderate to severe CTS that does not respond to non-surgical options. Currently in the United States, 500,000 carpal tunnel release surgeries are performed each year.

Traditionally, carpal tunnel release surgery has been performed using an open technique, which involves making a 1-inch incision in center of the palm distal to the volar wrist crease and releasing the transverse carpal ligament to relieve pressure on the median nerve. It is estimated that about 90% of carpal tunnel release surgeries utilize such an open technique, commonly because that technique presents a lower risk of median nerve injury. Downsides of an open surgical technique include longer recovery time, longer time away from work, and scar tenderness on the palm for several months.

In recent years, minimally invasive techniques have become more popular as an alternative to open surgery techniques. The benefits of minimally invasive carpal tunnel release include shorter recovery time, faster return to work, and less scar tenderness compared to open surgery techniques. For example, patients are usually able to return to work and normal activities a few weeks sooner after minimally invasive carpal tunnel release procedures than would be expected after open surgical procedures.

However, as with any surgical procedure, there are potential complications associated with minimally invasive carpal tunnel release. These can include nerve or blood vessel injury, infection, persistent pain, and recurrence of symptoms. Indeed, with current minimally invasive repair options, the risk for median nerve injury and incomplete release of the transverse carpal ligament still exists. Any injury to the median nerve or its branches is irreversible and considered a severe complication. Incomplete release of the carpal tunnel will most likely worsen the patient's symptoms. Most hand surgeons still prefer to perform an open carpal tunnel release technique to avoid those risks.

Accordingly, what is needed is an effective and safe minimally invasive carpal tunnel release device and method that can ensure proper release of the transverse carpal ligament to relieve pressure on the median nerve without risk of damage to the median nerves or any tendons. What is further needed is a carpal tunnel release device and method that can be used in a simple and expedient manner and ensure safety of the procedure regardless of any potential anatomic challenges. Still further, what is needed is a carpal tunnel release device and method that can position the device within the carpal canal proximate to the transverse carpal ligament with a high degree of certainty and locate a blade with precision to sever the ligament, and only the ligament, in a minimally invasive manner. Accordingly, it is the general aim of the present invention to provide an improved, minimally invasive carpal tunnel release device and method that is easy to position and use during surgery that overcomes the problems and drawbacks associated with prior art carpal tunnel repair techniques, and therefore significantly improves the utility, efficiency, efficacy and safety of such minimally invasive repair techniques.

### SUMMARY OF THE INVENTION

The present invention is generally directed to a minimally invasive carpal tunnel release device and method capable of ensuring complete release of the transverse carpal ligament to relieve pressure on the median nerve without risk of damage to the median nerve or any tendons in the wrist. Indeed, the device of the present invention is designed to eliminate any possibility of median nerve injury.

The minimally invasive carpal tunnel release approach in accordance with the present invention generally involves making a small incision away from the palm of the hand, proximal to the volar wrist crease and releasing the transverse carpal ligament from proximal to distal fashion (anterograde release) using specific instrumentation inserted through the incision and directed into the carpal tunnel.

The device generally comprises an outer cannula and an inner cannula insert which are collectively inserted into the transverse incision approximately 1 cm proximate to the volar wrist crease between the palmaris longus and flexor carpi ulnaris tendons. In accordance with an embodiment of the present invention, the outer cannula has a closed end and a dorsal groove that, once the device is properly positioned in the wrist and the inner cannula insert is removed, receives the transverse carpal ligament. Within the wrist, the groove is positioned to be facing upwards (i.e., towards the patient's palm) so that when the inner cannula insert is removed, the ligament will sit in the groove. The design of inner cannula insert helps to strengthen and add rigidity to the device during placement of the device into the carpal canal and facilitate the cannula insertion. The inner cannula insert further includes a blade surface, which may be fixed or retractably deployed, and which is used to sever the transverse carpal ligament in accordance with the minimally invasive procedure of the present invention. In preferred operation, once the device is placed within the patient's wrist to a desired location, the inner cannula insert is first removed so that the transverse carpal ligament can sit in the dorsal groove of the outer cannula. Once the transverse carpal ligament is sitting properly in the outer cannula dorsal groove, the inner cannula insert is pushed back in towards the distal end of the device to cut the ligament. The blade surface at the end of inner cannula is deployed and advanced distally to sever the ligament.

In accordance with preferred embodiments of the present invention, the dorsal groove in the outer cannula has the dimensions to receive and accommodate the transverse carpal ligament once the inner cannula insert is pulled back. When the inner cannula insert is reinserted into the outer cannula, there is no risk of the blade severing anything other than what is seated within the dorsal groove of the outer cannula. That is, the risk of damage to the median nerve or any other nerves or tendons in the wrist is eliminated. In preferred embodiments, the inner cannula insert fits completely within the cross-sectional area of the outer cannula so there is little risk of the blade severing anything outside of the outer cannula's volume (such as the median nerve or similarly located tendons) as the inner cannula insert if being moved within the outer cannula.

The complete release of the transverse carpal ligament can be confirmed by direct visualization using an arthroscopy camera placed inside the outer cannula. In alternate embodiments, a camera can be inserted through the outer cannula (once the inner cannula insert is removed), or through a slot provided in the inner cannula insert. The device and method of the present invention can further be assisted by camera or ultrasound.

In alternate embodiments of the present invention, the device can include gradient scale and/or a dorsal probe, for example, on the exterior surface of the outer cannula to aid in properly positioning and locating the dorsal groove proximate the transverse carpal ligament. In accordance with the preferred methodology of the present invention, the aim is to position the dorsal groove exactly under the transverse carpal ligament and facing upward so that the ligament is properly seated within the groove when the inner cannula insert is removed. Then, once the inner cannula insert is reintroduced, it will cut only the ligament seated within the dorsal groove. The dorsal probe is provided to palpate the transverse carpal ligament as the device is being inserted into the carpal tunnel. The probe helps to identify the distal border of the ligament and ensure the proper positioning of the cannula. Additionally, the gradient scale can be provided on the exterior of the outer cannula and/or along the length of the inner cannula insert, or on an external guide attached to the outer cannula, to accurately gauge the location of the dorsal groove and/or the extent of deployment of the inner cannula insert within the outer cannula.

As noted, carpal tunnel syndrome is a common nerve disorder that can be treated with a range of conservative and invasive options. Minimally invasive carpal tunnel release utilizing the present invention is a newer technique that offers potential benefits in terms of reduced pain and faster recovery. Since most of the current systems utilized still have the potential to cause injury to the median nerve, eliminating that risk would make hand surgeons more comfortable with using minimally invasive techniques and provide a great benefit for carpal tunnel syndrome patients. The present invention provides a device and method of use that eliminates the risk of damage to the median nerve.

These and other objects, features and advantages of the present invention will become apparent in light of the detailed description of embodiments thereof, as illustrated in the accompanying drawings.

### BRIEF DESCRIPTION OF THE DRAWINGS

FIG. 1 illustrates a perspective view of a first embodiment of an outer cannula for use in the minimally invasive carpal tunnel release device in accordance with the present invention.

FIG. 2 illustrates a different perspective view of the outer cannula of FIG. 1.

FIG. 3 illustrates a perspective view of a first embodiment of an inner cannula insert for use in the minimally invasive carpal tunnel release device in accordance with the present invention.

FIG. 4 illustrates a different perspective view of the inner cannula insert of FIG. 3.

FIGS. 5a-5c illustrate planar top, side and end views of the outer cannula of FIG. 1.

FIGS. 6a-6c illustrate planar bottom, side and end views of the inner cannula insert of FIG. 3.

FIGS. 7a and 7b illustrates blade deployment of the inner cannula insert relative to the dorsal groove of the outer cannula.

FIGS. 8-10 illustrate deployment of the minimally invasive carpal tunnel release device in accordance with the present invention within the patient's wrist. FIG. 8 illustrates insertion of the device into the wrist so that the dorsal groove of the outer cannula is located proximate the transverse carpal ligament and subsequent removal of the inner cannula insert. FIG. 9 illustrates the removed inner cannula insert and subsequent seating of the transverse carpal ligament in the dorsal groove. FIG. 10 illustrates reinsertion of the inner cannula insert into the outer cannula, and corresponding deployment of the blade surface for cutting the transverse carpal ligament.

FIG. 11 illustrates a perspective view of an alternate embodiment of an outer cannula for use in the minimally invasive carpal tunnel release device in accordance with the present invention.

FIG. 12 illustrates a perspective view of an alternate embodiment of an inner cannula insert for use in the minimally invasive carpal tunnel release device in accordance with the present invention.

FIG. 13 illustrates a perspective view of an alternate embodiment of the outer cannula of FIG. 1 incorporating an external guide attached thereto.

FIG. 14 illustrates a planar side view of the combined outer cannula and external guide of FIG. 13.

FIG. 15 illustrates a perspective view of the combined outer cannula and external guide of FIG. 13 with the exterior guide removed.

### DETAILED DESCRIPTION OF PREFERRED EMBODIMENTS

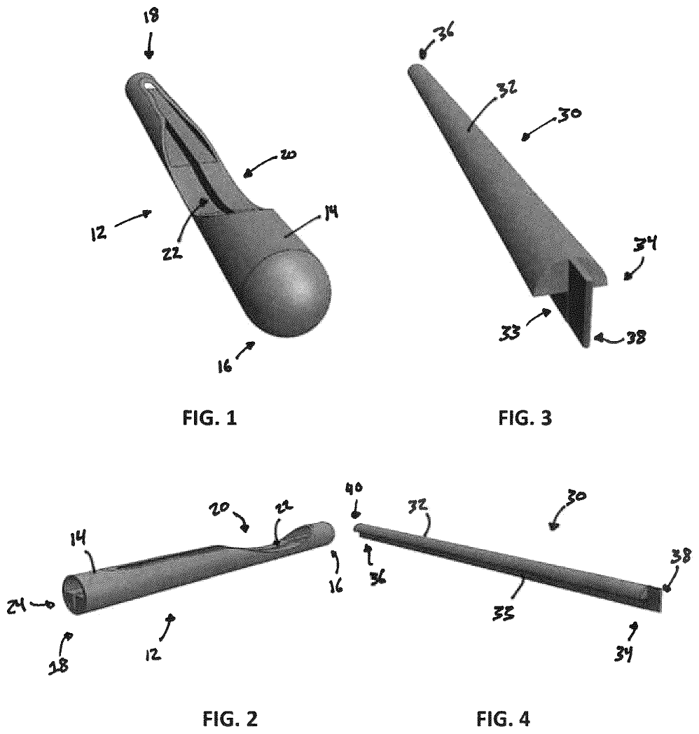

Embodiments of a minimally invasive carpal tunnel release device in accordance with the present invention is illustrated in FIGS. 1-7 and generally designated by reference numeral 10. In general, the device 10 comprises an outer cannula 12 and an inner cannula insert 30 designed to be inserted into the outer cannula 12. In intended operation, the device 10 is designed to be deployed into a patient's wrist to the carpal canal or carpal tunnel for releasing the transverse carpal ligament to ease the pain and pressure exerted on the median nerve in the wrist due to complications of CTS. In accordance with the present invention, the device 10 is inserted with the inner cannula insert 30 pre-inserted into the outer cannula 12. The outer cannula 12 and the inner cannula insert 30 are collectively inserted into a transverse incision approximately 1 cm proximate to the volar wrist crease between the palmaris longus and flexor carpi ulnaris tendons. The device 10 is designed to be flexible for insertion through the incision in the patient's wrist as well as movement through the forearm and wrist until properly positioned. However, the inner cannula insert 30 adds necessary strength and rigidity to the outer cannula 12 so that the device 10 can be moved through the patient's forearm and wrist without being impeded.

Referring to FIGS. 1-2, the outer cannula 12 is illustrated and comprises a body 14 defining an interior longitudinal cavity. The body 14 defines a closed distal end 16 that is preferably rounded or hemispherical to aid in movement of the device 10 through the patient's forearm and wrist, and also an open proximal end 18 through which the inner cannula insert 30 can be inserted and from which said insert 30 can be removed. The outer cannula 12 further includes a dorsal groove 20 generally comprising a recessed portion of the body surface. As described in more detail below, the dorsal groove 20 is designed to receive and hold the transverse carpal ligament once the device 10 is properly positioned for releasing said ligament from exerting unwanted pressure on the patient's median nerve. Within the wrist, the dorsal groove 20 is positioned to be facing upwards (i.e., towards the patient's palm) so that when the inner cannula insert 30 is removed, the transverse carpal ligament will sit in the dorsal groove 20.

Still referring to FIG. 1, the outer cannula 12 further includes an interior groove 22 axially extending along the length of the interior longitudinal cavity of the outer cannula 12 that is adapted to receive and guide the inner cannula insert 30. Referring to FIG. 2, the open end 18 of the outer cannula 12 includes an end stop 24 for restricting longitudinal movement of the inner cannula insert 30.

Referring to FIGS. 3-4, the inner cannula insert 30 comprise a body 32 having a complementary shape to the interior longitudinal cavity of the outer cannula 12. In this regard, the body 32 of the inner cannula insert 30 includes an axially extending longitudinal projection 33 adapted to fit into the internal groove 22 of the outer cannula 12. The inner cannula insert 30 is designed to add strength and rigidity to the outer cannula 12 when inserted into the patient's wrist to facilitate insertion and movement of the device 10 into and through the patient's carpal canal. The inner cannula insert 30 defines a distal end 34 having a blade projection 38 for cutting the transverse carpal ligament during intended use of the device 10, and a proximal end 36 having a stop 40 for restricting longitudinal movement of the inner cannula insert 30 when it engages the end stop 24 on the outer cannula 12. Though shown as a fixed blade 38, the inner cannula insert 30 can include a retractable blade projection without departing from the principles and spirit of the present invention.

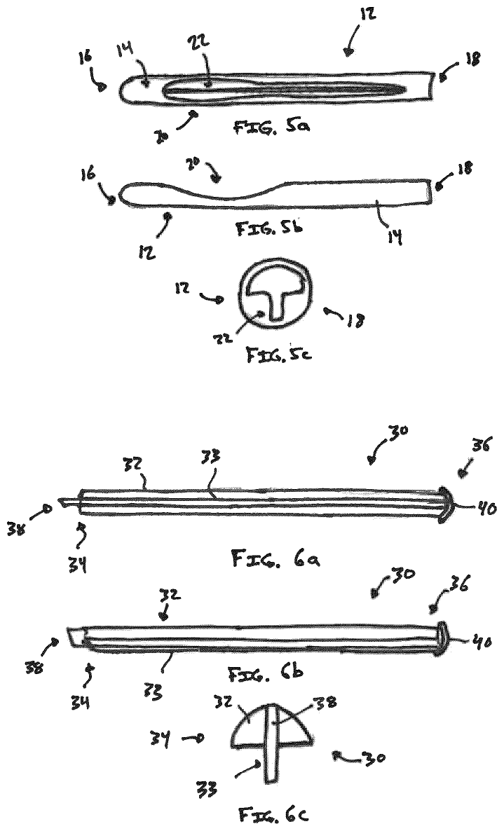

FIGS. 5a-5c respectively illustrate planar top, side and axial proximal end views of a preferred embodiment of the outer cannula 12 for use in the device 10 of the present invention. A preferred design of the dorsal groove 20 is apparent in the top and side views of FIGS. 5a and 5b. The cross-sectional shape of the interior longitudinal cavity, shown in FIG. 5c, is adapted to receive the inner cannula insert 30. More preferably, it is desirable for the inner cannula insert 30 to fit snugly within this cavity so that the deployment and positioning of the inner cannula insert 30 within and relative to the outer cannula is precisely controlled. In this regard, the inner cannula insert 30 is designed to have a cross-sectional shape that complements the cross-sectional area defined by the interior longitudinal cavity of the outer cannula 12. For example, the interior longitudinal cavity (as shown in FIG. 5c) defines the longitudinal interior groove 22, which receives the longitudinal projection 33 provide on the inner cannula insert 30 (and shown in cross-section in FIG. 6c). The complementary shapes help keep the inner cannula insert 30 straight relative to the outer cannula 12 during deployment and reduces the risk of the blade projection 38 thereon from severing anything but the desired transverse carpal ligament.

FIGS. 6a-6c respectively illustrate planar bottom, side and axial distal end views of a preferred embodiment of an inner cannula insert 30 for use in the device 10 of the present invention. As shown in FIG. 6c, the cross-sectional area of the inner cannula insert 30 is designed to fit snugly within the cross-sectional space defined by the interior longitudinal cavity formed within the outer cannula 12. More particularly, the longitudinal projection 33 of the inner cannula insert 30 is designed to fit within the longitudinal interior groove 22 to precisely guide the inner cannula insert 30 within the outer cannula 12 especially when deploying the blade projection 38 to sever the transverse carpal ligament.

FIGS. 7a and 7b illustrate the deployment of the blade projection 38 on the inner cannula insert 30 once the device 10 is positioned in the patient's wrist and the transverse carpal ligament is properly seated in the dorsal groove 20 of the outer cannula 12. In accordance with the methodology of the present invention, the blade projection 38 is designed to cut the ligament seated in the dorsal groove 20 and only that ligament. Any other anatomical structure outside the outer cannula 12 and away from the dorsal groove 20, including but not limited to the median nerve and flexor tendons, will not be cut by the blade projection 38, as the inner cannula insert 30 is kept within the cross-sectional area of the outer cannula 12 and along the path of the internal groove 22, as shown in FIGS. 7a and 7b. Further, deployment of the blade projection 38 is restricted by the stop 40 on the inner cannula insert 30 being limited by the end stop 24 provided on the proximal end 18 of the outer cannula 12. Still further, positioning of the device 10 and the extent of deployment of the inner cannula insert 30 can be facilitated and moreover determined with a high level of precision by using a dorsal probe and/or a gradient or other markings, such as on the exterior of the outer cannula 12 and/or along the length of the inner cannula insert 30, and/or on an external guide, such as illustrated in FIGS. 13-15 and described in more detail below.

Referring to FIG. 7a, the initial deployment of the inner cannula insert 30 within the outer cannula 12 is for insertion of the device 10 into the patient's wrist. The inner cannula insert 30, as deployed within the outer cannula, provides rigidity to the device 10 to aid in maneuvering the device 10 through the patient's wrist—i.e., to the carpal canal—so as to locate the dorsal groove 20 proximate to the transverse carpal ligament. In this set-up, the inner cannula insert 30 can be reversed so that the proximal end 36 is projecting towards the closed distal end 16 of the outer cannula 12. Once the device 10 is positioned in its desired position—i.e., with the dorsal groove 20 aligned with the transverse carpal ligament—the inner cannula insert 30 is removed so that the transverse carpal ligament can be seated in the dorsal groove 20. Then, the inner cannula insert 30 can be flipped around and reinserted so that the blade projection 38 on the distal end 234 of the inner cannula insert is deployed into the outer cannula 12 towards the closed distal end 16 thereof, as illustrated in FIG. 7b, to sever the seated transverse carpal ligament.

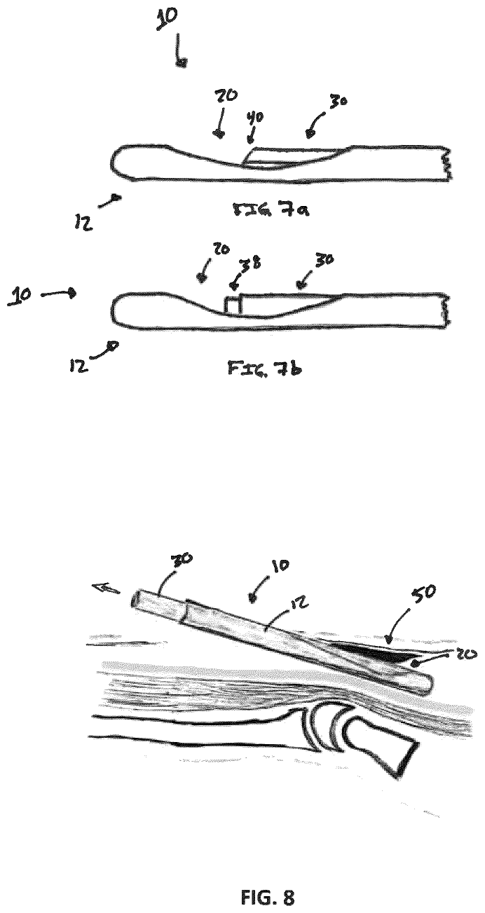

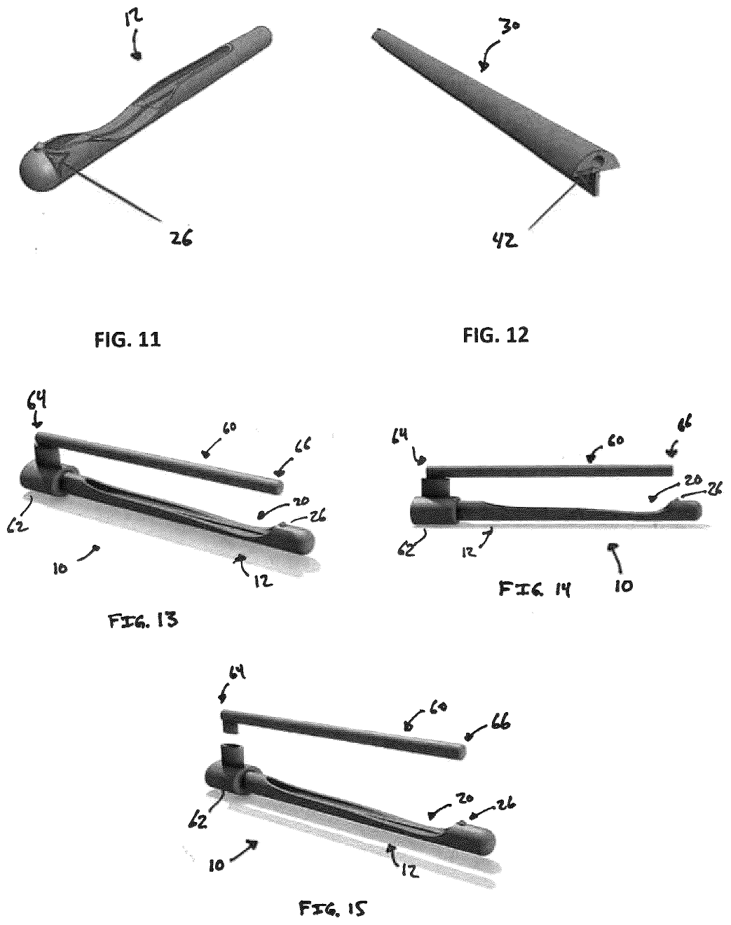

FIGS. 8-10 further illustrate deployment of the minimally invasive carpal tunnel release device 10 in accordance with the present invention within the patient's wrist. FIG. 8 illustrates insertion of the device 10 into the incision in the patient's wrist so that the dorsal groove 30 of the outer cannula 12 is located proximate the transverse carpal ligament (generally depicted as reference numeral 50). The device 10 can also include a gradient scale on the outer cannula 12 and/or along the length of the inner cannula insert 30 (not shown), and/or on an eternal guide (as shown in FIGS. 13-15), and/or a dorsal probe (as shown in FIG. 11), provided, for example, on the exterior surface of the outer cannula 12, to aid in properly positioning and locating the dorsal groove 20 proximate the transverse carpal ligament 50. In accordance with the preferred methodology of the present invention, the aim is to position the dorsal groove 20 exactly under the ligament 50 and facing upward so that the ligament 50 is properly seated within the groove 20 when the inner cannula insert 30 is removed. In FIG. 8, the inner cannula insert 30 is deployed within the outer cannula 12 to provide rigidity to aid in maneuvering the device 10 into and through the patient's wrist. As further shown, the device 10 is slanted upon insertion. Once positioned, the inner cannula insert 30 is removed (along the indicated arrow) and the position of the outer cannula 12 can be adjusted as needed. In accordance with preferred embodiments of the present invention, the device 10 has some flexibility because of the dorsal groove 20. As noted, the inner cannula insert 30, when inserted within the outer cannula 12, imparts some strength and rigidity to the device 10 so that it can be inserted as desired.

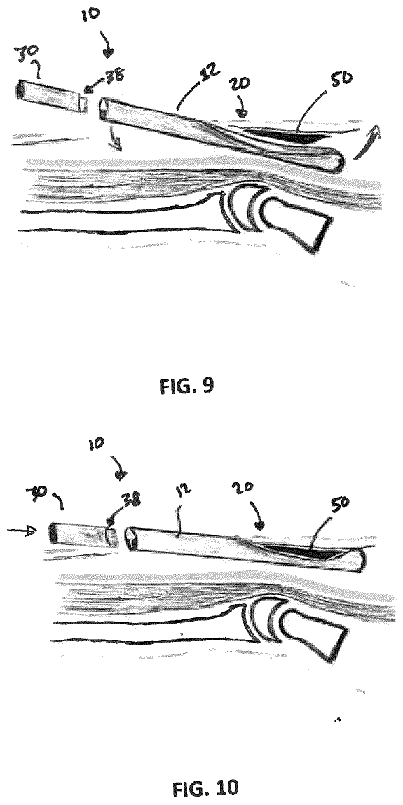

FIG. 9 illustrates removal of the inner cannula insert 30 and seating of the transverse carpal ligament 50 in the dorsal groove 20 of the outer cannula 12. Notably, after the inner cannula insert 30 is removed, the outer cannula 12 can be moved into place (along the depicted arrows) with the transverse carpal ligament 50 seated in the dorsal groove 30. After the outer cannula 12 is so positioned, the inner cannula insert 30 can be reintroduced into the interior longitudinal cavity of the outer cannula 12.

FIG. 10 illustrates reinsertion of the inner cannula insert 30 into the outer cannula 12 (along the depicted arrow), and corresponding deployment of the blade projection 38 for cutting the transverse carpal ligament 50. As noted, the inner cannula insert 30 can include a fixed blade surface 38 (as shown), or in the alternative, the inner cannula insert 30 can include a retractable blade surface that can be deployed once the inner cannula insert 30 has been reintroduced into the outer cannula 12. In operation, movement of the inner cannula insert 30 towards the closed distal end 16 of the outer cannula 12 will cause the blade projection 38 to engage and cut the transverse carpal ligament 50 seated in the dorsal groove 20.

Referring to FIG. 11, an alternate design for the outer cannula 12 is illustrated. In this embodiment, the distal end 16 of the outer cannula 12 is provided with a dorsal probe 26 for palpating the distal end of the transverse carpal ligament upon insertion of the device 10 into the carpal canal. In this regard, the dorsal probe 26 helps to identify the distal border of the ligament and ensure the proper positioning of the outer cannula 12 within the carpal canal.

Referring to FIG. 12, an alternate design for the inner cannula insert 30 is illustrated. In accordance with methods of the present invention, the use of the device 10 can be aided or checked using an arthroscopy camera or ultrasound. In FIG. 13, the inner cannula insert 30 is provided with a longitudinally extending channel or slot 42 through which an arthroscope can be inserted to visualize and confirm the transverse carpal ligament being released.

Referring to FIGS. 13-15, an external guide for aiding positioning of the minimally invasive carpal tunnel release device 10 within the patient's wrist is illustrated. As so illustrated, the external guide, generally designated herein as reference numeral 60, is removably attached to the outer cannula 12 via a collar 62 secured at the proximal end 18 of the outer cannula 12. When so attached, the external guide 60 projects generally parallel to and in the same directed as the outer cannula 12 and defines a proximal end 64 generally in line with the proximal end 18 of the outer cannula 12 and a distal end 66 projecting over the dorsal groove 20 in the outer cannula 12. As shown in more detail, the distal end 66 of the external device is precisely aligned with the dorsal probe 26 positioned as the distal end of the dorsal groove 20. In the alternative, the length of the external guide 60 can be the same as the length deployment of the inner cannula insert 30 within the outer cannula 12, so the surgeon can have a full understanding of how far the tip of the blade projection 38 will go into the wrist based on the position of the external guide 60. In this sense, the external guide 60, positioned outside the patient's wrist, provides an accurate visual depiction of the position of unseen components inside the patient's wrist.

The external guide 60 serves two key functions for a minimally invasive procedure. First, the external guide 60 allows the surgeon to precisely direct the device 10 to align with the third webspace of the patient's hand/wrist so as to properly position and orient the device 10 within the wrist proximate to the transverse carpal ligament. Second, the external guide provides a clear indication of the location of the dorsal groove 20 and the extent of deployment of the blade projection 38 on the inner cannula insert 30 relative to the dorsal groove 20. For example, the external guide 60 can include a gradient scale or other markings indicating the location of the outer cannula 12, the dorsal groove 20, and the inner cannula insert 30 relative to the outer cannula as it is deployed. Further, the markings on the external guide 60 can be coordinated with markings on the inner cannula insert 30 to determine the extent of deployment of the blade projection 38 when the inner cannula insert 30 is being inserted into the outer cannula 12.

Referring to FIG. 15, the external guide 60 can be removed, for example, once the device 10 is properly positioned and the extent of deployment of the inner cannula insert 30 and blade projection 38 is known with a high level of certainty. In the alternative, the device 10 could be inserted into the patient's wrist and then the external guide can be attached to the outer cannula 12 via the collar 62 to be able to determine the location of the dorsal groove 20 and the deployment position of the blade projection 38.

The collar 62 for the external guide 60 can further act as a handle to facilitate insertion and guidance of the device 10 into the patient's wrist.

The foregoing description of embodiments of the present invention has been presented for the purpose of illustration and description. It is not intended to be exhaustive or to limit the invention to the form disclosed. Obvious modifications and variations are possible in light of the above disclosure. The embodiments described were chosen to best illustrate the principles of the invention and practical applications thereof to enable one of ordinary skill in the art to utilize the invention in various embodiments and with various modifications as suited to the particular use contemplated.

What is claimed is:

1. A carpal tunnel release device comprising:
an outer cannula comprising a body defining an interior longitudinal cavity and having a dorsal groove formed into the body surface, wherein the dorsal groove is adapted to receive the transverse carpal ligament of a patient when the device is positioned in said patient's wrist proximate the carpal tunnel cavity; and
an inner cannula insert designed to be inserted into the interior longitudinal cavity of the outer cannula, said inner cannula insert including a blade projection adapted for cutting the transverse carpal ligament when said ligament is seated in the dorsal groove of the outer cannula.

2. The carpal tunnel release device according to claim 1, wherein the blade projection is fixed to a distal end of the inner cannula insert.

3. The carpal tunnel release device according to claim 1, wherein the blade projection comprises a retractable blade that can be deployed from a distal end of the inner cannula insert.

4. The carpal tunnel release device according to claim 1, wherein the body of the outer cannula includes an interior groove axially extending along the length of the interior longitudinal cavity of the outer cannula, and
wherein the inner cannula insert includes an axially extending longitudinal projection adapted to fit into the internal groove of the outer cannula.

5. The carpal tunnel release device according to claim 1, further comprising an external guide removably attached to the outer cannula and projecting generally parallel to said outer cannula, wherein when said outer cannula is positioned in the patient's wrist, the external guide is projected outside and over the patient's wrist.

6. The carpal tunnel release device according to claim 5, wherein the external guide includes at least one marking indicating the extent of deployment of the inner cannula insert when said inner cannula insert is inserted into the outer cannula.

7. The carpal tunnel release device according to claim 6, wherein the at least one marking comprises a gradient scale provided on the external guide.

8. The carpal tunnel release device according to claim 1, wherein the body of the outer cannula has an open proximal end providing access for the inner cannula insert into the interior longitudinal cavity and a closed distal end restricting the longitudinal movement of the inner cannula insert within the outer cannula.

9. The carpal tunnel release device according to claim 8, further comprising a probe positioned on the distal end of the outer cannula for palpating the distal end of the transverse carpal ligament upon insertion of the device into the carpal canal of the patient.

10. The carpal tunnel release device according to claim 1, wherein the inner cannula insert further includes a longitudinally extending channel.

11. The carpal tunnel release device according to claim 10, further comprising an arthroscope adapted to be inserted through the longitudinally extending channel in the inner cannula insert.

12. A carpal tunnel release device comprising:
an outer cannula comprising a body defining an interior longitudinal cavity and having a dorsal groove formed into the body surface, wherein the dorsal groove is adapted to receive the transverse carpal ligament of a patient when the device is positioned in said patient's wrist proximate the carpal tunnel cavity;
an inner cannula insert designed to be inserted into the interior longitudinal cavity of the outer cannula, said inner cannula insert including a blade projection adapted for cutting the transverse carpal ligament when said ligament is seated in the dorsal groove of the outer cannula; and
an external guide removably attached to the outer cannula and projecting in generally parallel alignment with said outer cannula, wherein when said outer cannula is positioned in the patient's wrist, the external guide is projected outside and over the patient's wrist, wherein the external guide includes at least one marking facilitating determining the position of the dorsal groove within the patient's wrist and/or the extent of deployment of the inner cannula insert within the outer cannula.

13. The carpal tunnel release device according to claim 12, wherein the at least one marking comprises a gradient scale provided on the external guide.

14. The carpal tunnel release device according to claim 12, wherein the blade projection is fixed to a distal end of the inner cannula insert.

15. The carpal tunnel release device according to claim 12, wherein the blade projection comprises a retractable blade that can be deployed from a distal end of the inner cannula insert.

16. The carpal tunnel release device according to claim 12, wherein the body of the outer cannula includes an interior groove axially extending along the length of the interior longitudinal cavity of the outer cannula, and
wherein the inner cannula insert includes an axially extending longitudinal projection adapted to fit into the internal groove of the outer cannula.

17. The carpal tunnel release device according to claim 12, wherein the body of the outer cannula has an open proximal end providing access for the inner cannula insert into the interior longitudinal cavity and a closed distal end restricting the longitudinal movement of the inner cannula insert within the outer cannula.

18. The carpal tunnel release device according to claim 17, further comprising a probe positioned on the distal end of the outer cannula for palpating the distal end of the transverse carpal ligament upon insertion of the device into the carpal canal of the patient.

19. The carpal tunnel release device according to claim 12, wherein the inner cannula insert further includes a longitudinally extending channel.

20. The carpal tunnel release device according to claim 19, further comprising an arthroscope adapted to be inserted through the longitudinally extending channel in the inner cannula insert.
